(19) United States
(12) Patent Application Publication
(10) Pub. No.: US 2010/0022969 A1
(43) Pub. Date: Jan. 28, 2010

(54) PERCUTANEOUS GASTROSTOMY CATHETER WITH INFLATABLE BALLOON AND WITH BIODEGRADABLE ANCHORAGE MEANS

(76) Inventor: Serge Renaux, Lamalou les Bains (FR)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/375,018

(22) PCT Filed: Jul. 31, 2007
(86) PCT No.: PCT/FR07/01319

§ 371 Date: Jan. 25, 2009

(51) Int. Cl. A61M 25/00

(52) U.S. Cl. 604/264

(57) ABSTRACT

The invention relates to a percutaneous gastrostomy probe comprising a tube (1), a means of internal retention (2) and an external collar (3). The probe according to the invention is characterized essentially in that the means of internal retention (2) consists of a non-biodegradable inflatable balloon (21) connected to separate anchorage means (22) held in place by threads (23) sutured to external points (24) and made entirely of a biodegradable polymer or copolymer such that the nature of the polymers employed, their dosage and their number molar mass are determined in such a way as to obtain a connection which maintains its mechanical properties of retention until the stomach and abdominal walls have adhered together.

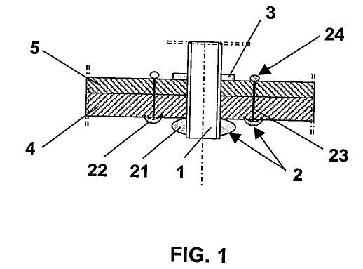

### FIELD OF INVENTION

The present invention relates to percutaneous endoscopic gastrostomy (PEG) and radiological percutaneous gastronomy (RPG) catheters enabling direct access to the gastric cavity for enteral feeding.

### BACKGROUND TECHNOLOGY

Percutaneous gastrostomy is currently the standard route for prolonged enteral feeding. Due to the simplicity and speed of technology and the development of the equipment, gastroenterologists or radiologists are increasingly sought, and the placement is accessible to any endoscopist or radiologist.

There are two placement techniques:
the endoscopic <<pull>> technique, mainly used by gastroenterologists: the sterile PEG kits generally include a puncture trocar, a double strand of wire, a tubular gastrostomy catheter, a flange-type internal means of retention, an external fixation flange: ideally, crocodile or rat tooth grasping forceps are used to grasp the intragastric wire. A diathermic cove or biopsy forceps can also be used;
the <<push>> technique via the abdominal wall, used mainly by radiologists: in this case anchors enable binding of the stomach to the abdominal wall for the time required for the formation of adhesions between the external part of the stomach and the abdominal wall. A catheter balloon is then positioned through the abdominal wall using a dilator and a peel-off nozzle.

The catheters are generally made of silicone or polyurethane, inert and well tolerated materials. Various sizes or Charrières are available, the small catheters being blocked more easily.

There are extractable and non-extractable catheters.

Non-extractable catheters, to be replaced, should be cut flush with the cutaneous orifice. The internal device is then pushed into the stomach.

The internal means of retention can be retrieved by endoscopy, an operation that can turn out to be delicate, or evacuated via the natural routes with risks of obstruction and intestinal perforation.

The advantage of non-extractable catheters lies in their relatively rigid internal flange thus resisting an attempt of pulling out by an agitated restless patient.

The extractable catheters have a retractable removable internal flange, or a deflatable retention system, enabling their removal via the cutaneous orifice by firm pulling.

The advantage of these flexible systems is to be able to pass through a severe stenosis, avoiding endoscopy, but they have less resistance to pulling out.

The choice between extractable and non-extractable catheters, made of silicone or polyurethane, depends on the indication, taking into account the advantages and disadvantages of each type of catheters.

The extractable catheters made of silicone are suitable for temporary enteral feeding.

Non-extractable or polyurethane catheters are more suitable for permanent enteral feeding or a restless patient.

The replacement of the gastrostomy catheters may be needed in case of obstruction, tube deterioration (cracking, porosity, expansion, colonization by candida).

The majority of replacement devices are catheters with a water-inflatable balloon, made of silicone. Their suitability for the gastric environment and an external retention flange enables safe use.

There is also a shorter gastrostomy button and on bare skin which, because of its aesthetic advantage and comfort, is indicated in the young or outpatient subject. It cannot be installed initially except with difficulty, however, and comes most often as replacement for a catheter already positioned in the stomach.

The catheter according to the invention is the type including:

a) a tubing designed to pass through the stomach and abdominal walls of the subject,

b) an internal means of retention, of the inflatable balloon type, associated with means of anchoring, designed to be joined to the aforementioned tubing and to be maintained pressed against the internal face of the stomach wall,

c) an external flange, traversed by the aforementioned tubing, designed to be pressed against the external face of the abdominal wall and exert, in cooperation with the internal means of retention, a pressure adapted to press the stomach wall against the abdominal wall in the area of the stoma.

### SUMMARY OF INVENTION

The invention aims to realize a catheter of the type in question designed to implement a new and original solution to eliminate the drawbacks mentioned above.

To that end, it relates to a percutaneous endoscopic gastrostomy catheter, which is essentially characterized in that the internal means of retention is constituted by an inflatable balloon, non-biodegradable, associated with distinct means of anchoring maintained by threads, sutured in external points, made entirely of a biodegradable copolymer or polymer, of which the nature of polymers used, their amount, and their molar mass number, are determined to obtain a connection that retains its mechanical retention properties until the adhesion of stomach and abdominal walls between themselves.

The polymer or copolymer used is optimally chosen from among PLA and PLA GA structures.

The means of anchoring, which are constituted by small anchors, are hard, painful and traumatic for the subject. Hence the need to resorb them for, because of their fibrous encapsulation over time, they can not be evacuated through the natural routes.

The biodegradation of the means of internal anchoring constitutes one of the advantages of the invention, eliminating:
the risks of occlusion with fatal consequences;
the use of an endoscope, which requires anesthesia;
pain and trauma caused by the presence of the internal part of the means of anchoring.

The period of optimal healing of the stoma is approximately 21 days. Too short a period could lead to its poor healing with all the consequences that may arise mainly at the introduction of a replacement catheter.

### PRESENTATION OF FIGURES

The characteristics and advantages of the invention will emerge more clearly upon reading the following detailed description of at least one preferred implementation thereof given by way of non-limiting example and illustrated in the attached drawing (single FIGURE) which sectionally represents a partial view of a catheter provided with a balloon and a means of anchoring, sandwiching the stomach and abdominal walls.

### DETAILED DESCRIPTION OF THE INVENTION

The percutaneous gastrostomy catheter represented in the figures is the type including:
a tubing (1) designed to pass through the stomach (4) and abdominal (5) walls of the subject;
an internal means of retention (2) designed to be maintained pressed against the internal face of the aforementioned stomach (4);
an external flange (3), traversed by the tubing (1), designed to be pressed against the external face of the aforementioned abdominal wall (5) and exert, in cooperation with the internal means of retention (2), a pressure adapted to press the stomach wall (4) against the abdominal wall (5) in the area of the stoma.

The internal means of retention (2) is constituted by a non-biodegradable inflatable balloon (21) associated with distinct means of anchoring (22) maintained by threads (23), sutured in external points (24), made entirely of a biodegradable copolymer or polymer, of which the nature of polymers used, their amount, and their molar mass number, are determined to obtain a connection that retains its mechanical retention properties until the adhension of stomach and abdominal walls between themselves.

The tubing (1) is generally made of a non-biodegradable biocompatible material such as, for example, silicone or polyurethane.

The choice of biodegradable copolymer or polymer suitable for the considered medical application was the subject of tests including synthesizing various polymers and copolymers into samples, immersed in a model of gastric fluid, with dimensions similar to those of the biodegradable internal means of retention concerned, capable of degrading within a well termined period (in particular between 1 and 3 months), having the physical and mechanical characteristics required, in terms of hardness or elasticity, shape changes, swelling due to the water, decomposition.

Tests were conducted on samples made with various polymers or copolymers of the types:
polylactic acid such as PLA 50 (Mₙ=21000 g/mol) and PLA 50 (Mₙ=46000 g/mol);
polylactic acid-glycolic acid such as PLA 37.5-GA 25 (Mₙ=39000 g/mol);
triblocks PLA-PEG (poly ethylene glycol)-PLA such as PLA 50-PEG (20000)-PLA 50 (Mₙ=277760 g/mol), PLA 50-PEG (20000)-PLA 50 (Mₙ=100600 g/mol), PLA 50-PEG (6000)-PLA 50 (Mₙ=56400 g/mol), PLA 96-PEG 12000-PLA 96 (Mₙ=68311 g/mol), PLA 96-PEG 8000-PLA 96 (Mₙ=71684 g/mol) and PLA GA-PEG-PLA GA.

The choice was made in the PLA class of polymers and more optimally, PLA GA to implement the anchoring means and the suture thread, where the shape memory problems do not arise.

Components having the effect of changing the mechanical characteristics and the rate of degradation, as well as body tolerance, can be added to the aforementioned copolymers.

Of course, the invention is not limited to the implementation modes described and illustrated for which the person of skill in the art will be able to provide other variations, particularly in the types of catheters employed and the materials constituting the subassemblies of the aforementioned catheters provided that the anchoring means and suture thread are completely biodegradable.

1-3. (canceled)

4. A percutaneous gastrostomy catheter, including:
a) a tubing configured to pass through the stomach and abdominal walls of a subject;
b) an internal means of retention configured to be maintained pressed against the internal face of the aforementioned stomach wall; and
c) an external flange, traversed by the tubing, configured to be pressed against the external face of the aforementioned abdominal wall and exert, in cooperation with the internal means of retention, a pressure adapted to press the stomach wall against the abdominal wall in the area of the stoma,
wherein the internal means of retention includes a non-biodegradable inflatable balloon associated with distinct means of anchoring maintained by threads, sutured in external points, made entirely of a biodegradable copolymer or polymer, of which the nature of polymers used, their amount, and their molar mass number, are determined to obtain a connection that retains its mechanical retention properties until the adhension of stomach and abdominal walls between themselves.

5. A catheter according to claim 4 wherein the polymer is the PLA type.

6. A catheter according to claim 4 wherein the copolymer is the PLA GA type.

7. A percutaneous gastrostomy catheter, including:
a) a tubing configured to pass through the stomach and abdominal walls of a subject;
b) an internal means of retention configured to be maintained pressed against the internal face of the aforementioned stomach wall; and
c) an external flange, traversed by the tubing, configured to be pressed against the external face of the aforementioned abdominal wall and exert, in cooperation with the internal means of retention, a pressure adapted to press the stomach wall against the abdominal wall in the area of the stoma,
wherein the internal means of retention includes a non-biodegradable inflatable balloon associated with an anchor maintained by threads, sutured in external points, made of a biodegradable copolymer or polymer, of which the nature of polymers used, their amount, and their molar mass number, are determined to obtain a connection that retains its mechanical retention properties until the adhension of stomach and abdominal walls between themselves.

8. A catheter according to claim 7 wherein the polymer is the PLA type.

9. A catheter according to claim 7 wherein the copolymer is the PLA GA type.
